(19) United States
(12) Patent Application Publication
(10) Pub. No.: US 2007/0178222 A1
(43) Pub. Date: Aug. 2, 2007

(54) Radiopaque coatings for polymer substrates

(76) Inventors: Daniel M. Storey, Longmont, CO (US); Terrence S. McGrath, Longmont, CO (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/542,557

(22) Filed: Oct. 3, 2006

Related U.S. Application Data
(60) Provisional application No. 60/763,262, filed on Jan. 30, 2006.

(51) Int. Cl. A61L 33/00

(52) U.S. Cl. 427/2.24

(57) ABSTRACT

Improved radiopaque coatings particularly suitable for polymer substrates are described. A modified ion plasma deposition (IPD) method is used to provide coatings with macroparticle-dense surfaces that have excellent radiopacity. The coatings are particularly adapted to polymer surfaces because of high adherence and resistance to peeling and flaking.

This application claims benefit of U.S. Provisional Application Ser. No. 60/763,262 filed Jan. 30, 2006, the entire contents of which are herein incorporated by reference.

### BACKGROUND OF THE INVENTION

1. Field of the Invention

The invention relates to radiopaque metal coatings on polymer substrates and in particular to improved radiopaque coatings on non-metal medical devices.

2. Description of Background Art

Visualization of internal organs and environment in the human body is important in diagnosis and selection of treatment methods. Minimally invasive procedures are performed through tiny incisions in the patient's body. Once inside the body, it is necessary to identify the target area for the procedure and be able to effectively treat that area.

Commonly used methods in current use are based on fluoroscopic examination. Fluoroscopes are x-ray devices equipped with a fluorescent screen on which the internal structures of an optically opaque object, such as the human body, may be continuously viewed as shadowy images formed by the differential transmission of x-rays through the object.

The use of gold as a radiopaque material is widespread. Application typically involves complex masking, seed layers, and expensive processing. Highly toxic and difficult to dispose of chemicals and time consuming vacuum processing can contribute to high costs. Gold is one of the more expensive metals with which to work, especially since the adhesion and waste recovery are limited.

In order to enhance the visibility of the internal body environment, there is an increasing demand for instruments that have a higher visibility on the fluoroscope, i.e., increased radiopacity, in order to determine more accurate treatment. Metal, because it is highly radiopaque, would be an ideal material for fluoroscopic examination; except that metals have limited flexibility and have a high risk of causing abrasion. Unfortunately, the plastics and polymers in current use are not sufficiently radiopaque to provide satisfactory results for x-ray based examinations.

Fixturing is frequently a problem in effectively coating surfaces that have complex shapes or are not electrically conductive. In most radiopacity applications, bands, stripes or other radiopaque markers are preferable to whole surface coverage. To manufacture these structures (and in turn saving money on expensive radiopaque material), complex fixturing that can carry electricity to a specific spot is necessary. Even if the current can be carried to a specific spot or band on a non-conductive medical device, a seed layer is needed to conduct the current at the spot where the coating is to be applied.

Application of a seed layer to non-conductive materials is generally a significant problem. This usually requires complex masking along with the use of a different deposition technique such as sputtering or ion beam assisted deposition (IBAD). This is a costly step leading to handling problems, increased cost due to the double processing and often results in poor coating adhesion.

Electroplating is currently the most commonly used process for coating medical devices; however, electrochemical manufacturing processes create significant caustic waste disposal issues. Gold and platinum are the only two radiopaque, biocompatible materials that can be easily electroplated to the needed thickness. Both these processes use caustic liquids such as cyanide that need to be disposed of in an environmentally acceptable manner. Even if the fixturing and seed layer problems can be overcome in a cost effective manner, post processing of toxic waste is not only expensive, but is also very difficult.

The need for radiopaque coatings in the medical device market is well known. Tracking, placement, measurement, scale, and location are some of the necessary components that are lacking in modern minimally invasive surgeries. Current technologies are limited to metal bands crimped onto catheters and expensive vacuum processing. The crimped metal band is becoming increasingly problematic and often unacceptable due to the high incidence of delimitation of the crimped band. Additionally, as the cost of healthcare rises, the cost effectiveness of coating non-metallic medical devices with expensive and poorly adhering coatings is diminished due to low profit margins.

Attempts have been made to apply radiopaque coatings to a range of substrates, such as plastics, polymers, and ceramics. A key problem in coating polymers with radiopaque materials is that most current state of the art processes, such as sputtered or electroplated coatings, have limited adhesion to flexible substrates or devices that require elasticity for medical use. Additional coating layers may be necessary to achieve acceptable adhesion despite the increase in cost due to increased processing time.

Plastic parts are not easily electroplated because plastic is a nonconductor of electricity. One approach to metallization is to use a series of steps in order to obtain reasonably adherent coatings. Plastics can be metallized but the steps are tedious and generally costly and several steps are required for effective plating. Initially, the plastic must be perfectly free of any oil, grease or any plastic injection mold compounds. If not properly cleaned, the metal will peel off over time from a plated plastic part. The part is next processed in a very aggressive chromic/sulfuric acid bath (not readily acceptable to the FDA for medical devices) to etch the plastic surface. The part is placed in a palladium chloride bath to allow metal particles to deposit in the pits made on the plastic surface. After the palladium metal deposition, the part can be electroplated with copper metal and then plated with chrome or other metals such as nickel or gold.

Current state of the art processes are limited in their ability to produce cost effective products that are safe for use in human or animal bodies, especially in the heart and arteries.

### SUMMARY OF THE INVENTION

The present invention is directed to a method for depositing radiopaque metal coatings on polymeric medical devices. The deposited coatings are thick enough to prevent x-ray transmission, yet do not affect polymer qualities such as flexibility which may interfere with medical procedures.

In one aspect, the method provides modified metal coatings for enhancing radiopacity of medical device components used for internal visualization or treatments. Coatings on nonmetal substrates that have improved adhesion and are radiopaque to x-radiation can be efficiently produced. Dense coatings on polymers and plastics have low visibility in fluoroscope and x-ray applications, thereby increasing the accuracy of placement and tracking in the human body.

Radiopacity, according to the present invention can be increased by controlling metal ion plasma deposition such that a relatively thin but highly dense coating of macro particles is formed on a polymer. This provides a radiopaque film that does not interfere with the flexibility required for manipulation in the body, while at the same time allowing use of x-radiation.

The present invention utilizes an IPD process modified to provide macroparticulate surface-layered radiopaque films on polymer surfaces. Not only have unexpected performance improvements been achieved, significant enhancement of radiopacity coatings has also been achieved, compared with electroplated films such as gold. The coatings can be deposited on flexible polymers other non-radiopaque materials such as ceramics, and on minimally radiopaque materials that require enhanced radiopacity. The disclosed IPD process has an extremely high volume output and is relatively low cost compared to other vapor deposition and electroplating methods.

Thus in addition to more economical methods and apparatus for coating manufacture, significantly improved radiopaque coatings are now possible for use on the flexible materials needed for manipulations in the human body.

Use of an IPD process provides radiopaque films that have a number of advantages over conventionally-produced radiopaque coatings and processes for depositing radiopaque coatings, such as electroplating which is slow and relatively costly.

It is difficult to obtain satisfactory adhesion of metals on plastics using other physical vapor deposition (PVD) processes, electroplating, or electro-less plating while keeping all the physical properties of the original substrate. For most metals deposited by these processes, adhesion is dependent on a strike layer of titanium or chromium and even then, tends to delaminate if the substrate is bent, twisted or stretched. Use of an IPD deposition imbeds a coating into a polymer so that peeling and flaking are virtually eliminated.

While electroplating and electro-less plating are relatively low temperature processes (less then 70° C.), most plasma vapor deposition processes require a pre-heat cycle and glow discharge, the pair usually resulting in temperatures exceeding 200° C. Most plastics melt well below this temperature. The IPD process can be preformed at a much lower temperature, allowing for low melting point plastics to be effectively coated without adversely affecting the original substrate specifications. Such low temperature deposition is achieved by controlling the deposition rate, especially the deposition of macro particles, which are produced in an IPD process. In general, higher macro particle deposition rates result in lower temperature depositions, while lower deposition rates result in higher temperature depositions. Macro particles are usually not charged and therefore do not induce a current on the substrate when deposited. In addition, the substrate spends less time in the plasma, so that little if any heating occurs.

Unlike traditional PVD and electroplating processes, the IPD-deposited radiopaque coatings can be scaled as necessary and still achieving a high throughput without sacrificing the quality and economy of coating desirable in the manufacture of medical devices.

The IPD process can be used to deposit metal coatings that normally would not provide acceptable radiopacity when deposited by traditional IPD methods. For example tungsten, molybdenum, and iridium have a higher radiopacity at comparable thicknesses to more expensive metals such as gold. Therefore, thinner coatings, using shorter processing times obtained with using the described IPD method achieve the same radiopaque results. This results in major cost savings and higher throughput, which is a significant advantage.

Typical plasma vapor deposition and electroplating are line of sight deposition and therefore it is difficult to coat complex devices without complicated fixtures. Even with the correct fixture, it may not be possible to evenly coat curved surface devices. The disclosed IPD process allows for non-line of sight coating that still maintains the radiopaque qualities without complicated fixtures, with coatings that readily conform to the part.

Highly radiopaque coatings can be produced from any metal that has an atomic number greater then 21, and a density greater then 4.5 g/cm², particularly Ti, Zr, Cr, Co, Ni, Mo, Pd, Ag, Hf, Ta, W, Ir, Pt, and Au; preferably Ag, Ti, Cu and Au. These metals can be deposited as thin films on polymer surfaces, making such highly radiopaque coatings ideal for use on catheters, valves, stents and particularly for implant devices that require flexibility.

Radiopaque coatings need not be limited to a single metal such as gold. Two different targets can be used so that an initial deposition can be made with one metal that covers the substrate surface with an adherent smooth surface, such as shown in FIG. 4, followed by a different metal that can be deposited in increasing amounts of macroparticles (a continuum of particulate size in the coating) or more discontinuously by immediately adjusting the arc speed and/or substrate position relative to the target. As an example, titanium can be deposited from a first target, followed by gold deposition from a second target in such a manner that a dense coating of gold macroparticles is formed immediately over the titanium or the gold coating is deposited with a gradual increase of macroparticles.

The invention is a new way for depositing a radiopaque coating on a polymer surface using an IPD process. First, a substantially macroparticle-free coating is deposited on the substrate followed by additional coating of macroparticles, which may be from a second target material. Preferably, the coating, while not homogeneous, appears as a single layer, and is deposited to a thickness of about 1 to about 100 microns. Thicker films are generally not desirable as radiopaque coatings.

The number and density of the deposited macroparticles can be determined by controlling distance of the substrate from the target and/or varying arc speed. The coating is preferably deposited continuously so that there are no discernable layers. Of course one may also deposit a first layer and a second layer so that each layer has a distinct homogeneous appearance. It is believed that this will not affect radiopacity characteristics so long as the coating surface is predominately populated with macroparticles. The macroparticles are preferably at least 1 micron in size; smaller microparticles may have less radiopacity.

The densities of macroparticles may range up to 90,000/cm²; and while this surface density provides excellent radiopacity properties for gold, no claim is made to an optimal density or that this density range is optimal for other metals. Nor has an optimal maximum size distribution been determined although it is believed that the ideal particle size will cluster in a range clustered around 1 micron, which is a “medium-size” macroparticle as defined herein.

Substrates may be of any desired material but polymer based substrates are particularly preferred because the majority of substrates where radiopacity surfaces are needed are in medical devices where visualization is important. Virtually any plastic substrate can be coated by the IPD method, including PTFE, ePTFE, polypropylene, polyester, PEEK, UHMWPE, silocone, polyimide and ABS. Polyimide and PEEK are preferred substrates as medical devices are often constructed of this material.

Examples of medical devices on which the described radiopaque coatings will be useful include valves, implants, catheters, stents and tubes. The radiopaque coatings are particularly preferred for used on PEEK spinal implants and catheters.

Suitable radiopaque coatings that can be produced using the IPD process include gold, titanium, niobium, molybdum and hafnium with gold being particularly preferred. Titanium may also be used, but is preferably used as an undercoat with gold macroparticles at least on the surface of the coating because gold normally provides better opacity than titanium.

Coating thickness are preferably in micron range. This thickness typically provides good radiopacity properties, but of course can be optimized depending on the coating metal. Preferable thicknesses for gold are in the 1-20 micron range, more preferably 1, 5, 10, 15 and 20 and most preferably in the 5 micron range.

An important aspect of the invention are the radiopaque films themselves. These films have unusual surfaces, comprising a dense macroparticle film surface over a substantially macroparticle-free adherent base undercoat. The coating may be homogeneous, controlled by deposition conditions, or heterogeneous by using discrete deposition conditions.

The coatings of the invention are desirable as radiopaque coatings on several types of medical instruments including stents, catheters, valves, tubes and implants. Coating thicknesses for practical use are preferably in the 1 micron to 100 nm range.

Definitions

Macros and macro particles refer to particles larger than a single ion. Small macro-particles refer to particles from two atoms to approximately 100 nanometers (also called nano-particles). Medium macro-particles refer to particles from 100 nanometers to about 1 micron. Large macro-particles refer to particles larger than 1 micron.

A radiopaque material does not allow passage or transmission of x-rays.

Fixturing is an important consideration in coating processes. Various types of substrate motion during the coating process can be effective in maximizing the homogeneity of the film. Each point on a fixed substrate has a different spatial relationship to the source when IPD processes are used. Mobile planetary substrate fixturing typically employs constant speed mechanisms with one or more degrees of freedom designed to average the target over large substrate areas to produce more uniform coatings.

Ion beam assisted deposition (IBAD) is used to densify sputtered coatings.

### BRIEF DESCRIPTION OF THE DRAWINGS

FIG. 1 is a sketch of the IPD apparatus: target material (1), substrate (2), mechanism for adjusting substrate distance from the target (3), vacuum chamber (4), power supply for the target (5).

FIG. 2 is another embodiment of the IPD apparatus; target (1), substrate (2) mechanism for adjusting substrate distance from the target (3), vacuum chamber (4), power supply for the target (5), and arc speed control (6).

FIG. 3 is a photograph showing an IPD deposited gold film on a plastic (polyimide) substrate, at 20× magnification on a total field of 253-262 microns. Deposition conditions were adjusted so achieve a high macroparticle density. The surface was calculated to have a macroparticle density of 90,000/cm² for macroparticles that are about 1 micron or larger in size.

FIG. 4 is a photograph showing the smooth surface texture of an IPD deposited gold film on a plastic (polyimide) substrate, at 20× magnification on a total field of 253-262 microns. Deposition conditions were controlled so that the surface was essentially free of macroparticles in the 1 micron or larger size range.

FIG. 5 illustrates an IPD apparatus with two targets that can be used simultaneously or serially; target A (1); target B (7); substrate (2); mechanism for adjusting substrate distance from target A or target B (3); vacuum chamber (4); power supplies for control of either target (5) and optionally an arc speed control for either target (6).

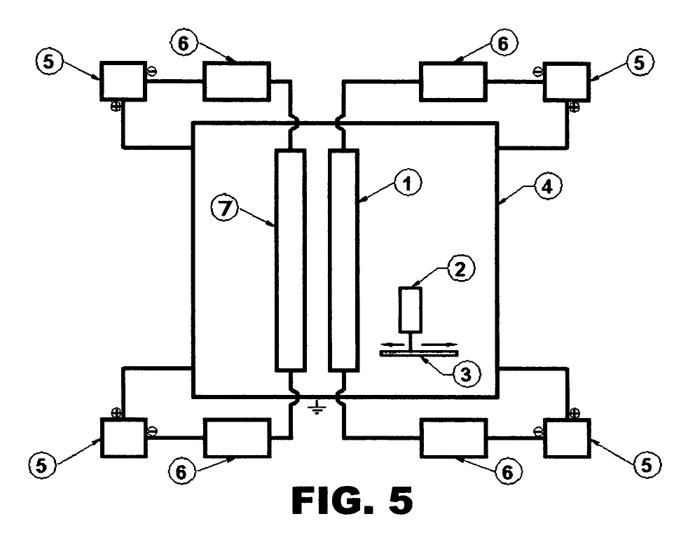

### DETAILED DESCRIPTION

A goal of the present work was to develop a method for producing a highly adherent, radiopaque film on polymer-based substrates. It was discovered that a controlled ion plasma deposition (IPD) process could produce enhanced adhesion of radiopaque coatings to polymers while also having higher deposition rates than other conventional radiopaque plating and deposition processes used in the industry. The excellent adhesion of the coatings has made it possible to deposit radiopaque coatings directly onto polymer substrates.

A surprising aspect of the new IPD method is that macro-particles ejected from the cathode (target) and deposed on the substrate actually enhance, rather than diminish the radiopaque quality of the metal coatings. While it is generally known that cathodic arc deposition processes can achieve higher deposition rates and tend to produce more macro-particles than other types of plasma deposition processes, it was unexpected that deliberately increasing macro-particle deposition would enhance radiopacity of IPD-deposited materials and that high quality films could be produced as thin films.

One aspect of the invention was the recognition that ion plasma deposition could be developed to be particularly well-suited to deposition of radiopaque coatings, not only because of the ability to deposit at high rates and achieve better adhesion, but also because of the effect of increasing macro-particle deposition. The invention is in part also directed to methods and apparatus for enhancing production and deposition of macro-particles to achieve dense, radiopaque coatings at high deposition rates with good adhesion characteristics. The coatings produced are dense, highly adherent, economical and are highly visible in low kilovolt (KV) x-ray ranges that are typically used in medical applications.

One feature of the new IPD method is the use of the distance/current relationship with target. The closer the substrate is to an arc source, the more macro-particles will be present on the substrate. As macro particles are ejected from the target, they evaporate so that the longer the time of flight, the more material is evaporated from the particle. Additionally, either a higher current or limiting the current to a level that occurs just before an arc split tends to cause more and larger macro particles.

A motorized unit that has the ability to move a substrate closer to and farther away from the target (cathode) can be used to initially deposit a fairly macro-free film for better adhesion on a substrate positioned far away from the target, which is then followed by deposition of a more macro particle dense film with the substrate positioned close to the target, which produces a more radiopaque film or coating. While such a motorized unit has not yet been made and used in this process, it is believed to be a fairly straight forward task that can be accomplished by persons skilled in the art, once they understand the principles of this invention.

The use of a controlled IPD power source, which can be configured to sufficiently slow (or accelerate) the speed of the arc is another feature of the invention. The traveling speed of the arc is directly related to the amount of macro particles produced. Essentially, slowing the speed of the arc on the surface of the target (cathode) will cause it to produce more macro particles, which can be used to increase the macro particle density, thus also the film density and the resulting radiopacity of the film. Conversely, increasing the speed of the arc on the cathode will decrease production of macro particles, thereby providing more high energy ions that can be embedded into the surface of the substrate to produce better adhesion. U.S. Pat. No. 6,936,145 describes a mechanical switch which is one possible means to increase and decrease travel speed of the arc. Such increase and decrease of arc speed results in the deposition (without internal movement) of a fairly macro-free film for adhesion, which can be followed directly by a macro dense film by manipulating the arc speed.

Other materials in addition to gold have been proposed as possible candidates for radiopacity due to their electronic configuration, large atomic cross section (higher atomic number in the periodic table) in the x-ray range and density. In addition to these characteristics, the coating material must be bio-compatible if used for radiopaque coatings on medical devices. Because of these requirements, tungsten, molybdenum, tantalum, and iridium will be useful for such coatings.

Typical coating rates achieved with the IPD process in this invention range from 100 nm to 5 microns per minute for materials such as gold or silver. Using the new IPD method, it is possible to coat over 45,000 square inches per hour at a coating rate of greater then 200 nm per minute. In addition to the increased coating rate and large volume, the IPD process required less handling per square inch due to the single layer coating, which translates to lower labor and higher processing rates/throughput.

### EXAMPLES

### Example 1—IPD Radiopaque Method

Ionic Plasma Deposition (IPD) utilizes a modified controlled cathodic arc discharge on a target material to create highly energized plasma. IPD differs from normal ion plasma depositions in several ways, including precise control of arc speed. This allows for faster movement, creating fewer macro particles without the use of sensors or filters, or slower movement, creating a greater amount and larger macro particles. It also gives the option of mixing the two modes to create a moderate amount of particles, or creating a near macro-free coating followed by a macro-dense coating. Alternatively, macroparticle density can also be controlled by adjusting movement of the substrate with respect to distance from the target during deposition.

Several nonmetal substrates have been coated with highly radiopaque coatings, including PTFE, ePTFE, polypropylene, polyester, PEEK, UHMWPE, silicone, polyimide, and ABS. The coatings deposited by the IPD method are highly adherent and typically have been found to imbed in polymer surfaces up to 100 nm, so that flaking and peeling are virtually eliminated.

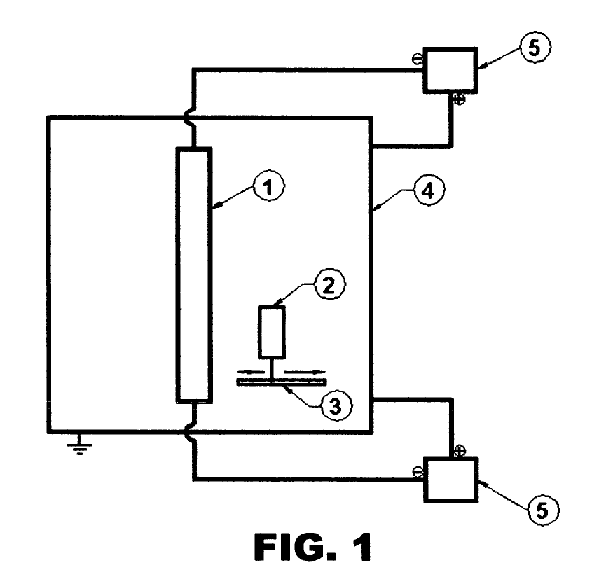

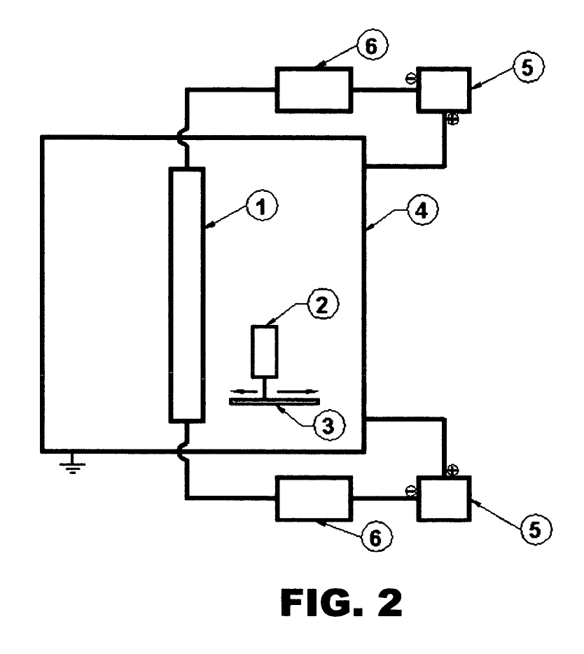

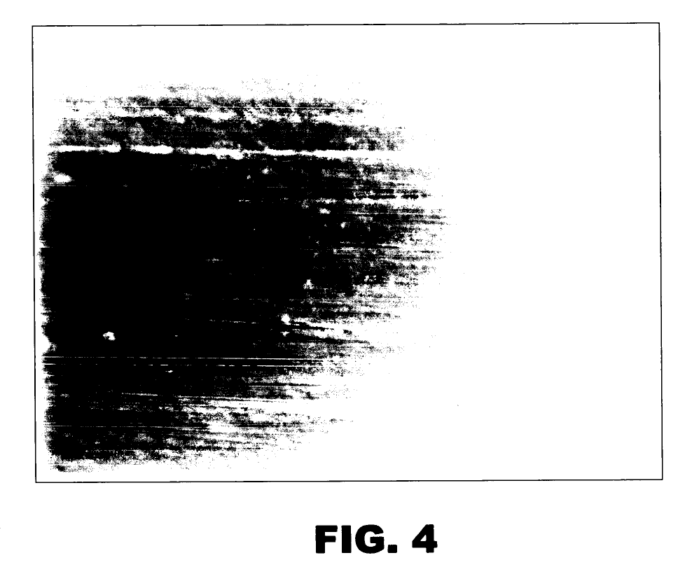

Radiopaque coatings were deposited using a modified IPD method. A typical apparatus is shown in FIG. 1 and FIG. 2 where either system provides control of the target metal deposition. Deposition conditions are adjusted to the size and type of substrate, the target material, which is typically gold or other metals commonly used for radiopaque films, and thickness of film desired. In preparing the films, a substrate, which can be as complex as a curved plastic tube, is placed at a distance from the target so that a metal/metal oxide smooth film is deposited uniformly over the surface. Thickness are preferably in the range of about 100 nm. FIG. 4 is a photograph at 20 fold magnification showing the surface appearance of a film deposited on a stainless steel substrate using an IDP apparatus as shown in FIG. 1 where the substrate was relatively far from a silver target, about 24 inches. Typical operating parameters are vacuum pressure of 0.1 mT to 30 mT, operating temperatures in the range of 25° C. to 75° C.

For purposes of obtaining a highly radiopaque film, deposition is preferably a continuous process where the deposited film characteristics are changed by either changing arc speed (FIG. 2) or adjusting position of the substrate in relation to the target (FIG. 1) so that larger particles, i.e., macroparticles are deposited. The surface of the film on a plastic (or metal) substrate will comprise a dense macroparticulate surface where the majority of the densely distributed particles are at least 1 micron in size. In order to initiate film deposition, a deposition of gold from the target was initiated at about 24 inches from the substrate until the substrate surface was coated. The substrate was then moved to about 8 in from the target, resulting in an increasing number of macroparticles being deposited.

The coating is preferably deposited as a continuous layer; i.e., two layers with distinct physical properties Macroparticle density throughout the film will increase from the surface of the substrate in relation to the speed with which the arc speed is changed and/or the substrate is moved in relation to the target. The final thickness of the coating can be controlled depending on the material deposited and a thickness that will provide a desired radiopacity for the intended use.

Radiopacity properties of a coating will be determined in part by its thickness and by the stopping power of the material, i.e., its ability to absorb and/or reflect x-rays. Atomic number, density and cross section all have an impact on the stopping power. Gold coatings with a thickness of 1 to 5 microns on a round substrate using the disclosed IPD method provide sufficient radiopacity for medical use.

### Example 2—Radiopacity of Gold on Polyimide

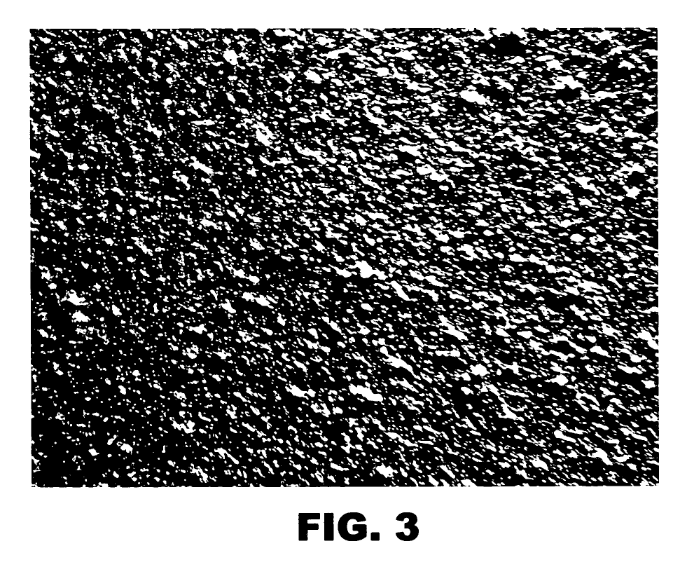

Samples of catheters were coated with 5, 10, 15, and 20 microns of radiopaque gold markers and tested in a conventional cath-lab system. A standard radiological procedure indicated an x-ray intensity of 60 kV and for large patients 90 kV was used. Under normal conditions (60 kV), the 10, 15, and 20 micron samples were visible. Using 90 kV, the 5 micron sample in addition to the 10, 15, and 20 micron samples were visible. The testing was performed with the prepared samples and no other biomass. The appearance of a typical gold film surface is shown in FIG. 3. The initially deposited gold has a smooth surface (FIG. 4) with few if any macroparticles.

### Example 3

### Radiopaque Coating of PEEK Spinal Implant

A spinal implant constructed of PEEK was coated with a 5 micron thick coating of gold using the IPD method described in example 1. The coating had an average of 100 nm macro-particles densely distributed over the coating surface. A typical macroparticle distribution of 90,000 cm² is shown in FIG. 3. The implant was masked such that when coated, only a limited area of the implant, typically not visible under x-ray irradiation, would be visible. This allows the medical professional implanting the device and any medical professional for decades, to see the orientation of the implant with great accuracy.

The coated portion of the implant was viewed with a fluoroscope at 60 kV and 90 kV with no other biomass. The fluoroscope imaged implant markings were highly visible.

### Example 4—Radiopacity of Gold Coating on PEEK Spinal Implant Overlaid with Mammalian Tissue

A spinal implant constructed of PEEK was coated with a 5 micron thick coating of gold deposited by the IPD method of Example 1. The coating had an average of 100 nm macro-particles densely distributed over the coating surface. The implant was masked such that when coated, only a limited area of the implant, typically not visible under x-ray irradiation, was visible. This allows the medical professional implanting the device, to see the orientation of the implant with great accuracy.

The coated part was illuminated with a fluoroscope at 60 kV and 90 kV with a one inch piece of pig flesh over the top of the implant. The resulting fluoroscope images showed that the implant markings were clearly visible, providing evidence that similarly coated implants will be visible through tissue.

What is claimed is:

1. An ion plasma deposition (IPD) method for depositing a radiopaque coating on a polymer substrate, comprising:
depositing a first substantially macroparticle-free coating on a polymer substrate;
depositing a second macrodense macroparticle coating over the first coating;
forming a coating to a thickness between about 1 micron to about 100 microns;
wherein arc speed or distance of the substrate surface from the IPD target controls macroparticle production and density.

2. The method of claim 1 wherein the first and second coatings are formed continuously without interruption of the deposition.

3. The method of claim 2 wherein macroparticle density increases in the deposited coating relative to variation of arc speed or substrate surface distance from the IPD target.

4. The method of claim 1 wherein the substantially macroparticle-free first layer is distinctly delineated from the densely distributed macroparticle second layer.

5. The method of claim 1 wherein the densely distributed macroparticle layer comprises about 90,000/cm² macroparticles 1 micron or larger in size.

6. The method of claim 1 wherein the plastic substrate is PTFE, ePTFE, polypropylene, polyester, PEEK, UHMWPE, silocone, polyimide or ABS.

7. The method of claim 1 wherein the substrate is polyimide or PEEK.

8. The method of claim 1 wherein the substrate is a medical device selected from the group consisting of valves, implants, catheters, stents and tubes.

9. The method of claim 7 wherein the substrate is a PEEK spinal implant.

10. The method of claim 1 wherein the substrate is a catheter.

11. The method of claim 1 wherein the radiopaque coating is gold, titanium, niobium, molybdum or hafnium.

12. The method of claim 11 wherein the radiopaque coating is gold or titanium.

13. The method of claim 11 wherein the radiopaque coating is gold.

14. The method of claim 11 wherein the gold is deposited at a thickness of 1, 5, 10, 15 or 20 microns.

15. The method of claim 14 wherein the gold is deposited at a thickness of 5 microns.

16. A polymer substrate coated with a radiopaque film comprising a densely distributed macroparticulate film surface over a substantially macroparticle-free adherent base coating continuous with the film surface.

17. The polymer substrate of claim 16 wherein the coating is between about 100 nm and about 1 micron thick.

18. The polymer substrate of claim 16 wherein the substrate is a device used in medical applications selected from the group consisting of stents, catheters, valves, tubes and implants.
